(19) United States
(12) Patent Application Publication
(10) Pub. No.: US 2005/0220868 A1
(43) Pub. Date: Oct. 6, 2005

(54) Policosanol composition and its use in treatment of hypercholesterolemia

(75) Inventor: William J. Lahl, Cuddebackville, NY (US)

Correspondence Address: OBLON, SPIVAK, MCCLELLAND, MAIER & NEUSTADT, P.C., 1940 DUKE STREET, ALEXANDRIA, VA, 22314, US

(73) Assignees: MARCOR DEVELOPMENT CORPORATION, Carlstadt, NJ (US); SHANGHAI FREEMEN INTERNATIONAL TRADING CO., LTD., Shanghai (CN)

(21) Appl. No.: 10/813,017

(22) Filed: Mar. 31, 2004

(51) Int. Cl. A61K 9/64; A61K 31/045

(52) U.S. Cl. 424/456; 514/724

(57) ABSTRACT

A composition is provided containing at least 90% of policosanols obtained from rice bran wax, wherein the policosanols contain from about 25 to about 35% of octacosanol, and its use in providing significant improvements in total cholesterol and/or LDL reduction in subjects in need thereof.

### BACKGROUND OF THE INVENTION

1. Field of Invention

The present invention relates to a policosanol composition containing octacosanol, that is useful for treatment of hypercholesterolemia, and is derived from rice bran wax.

2. Discussion of the Background

Cardiovascular disease, particularly heart disease and stroke, is a leading killer for both men and women in the United States, among all racial and ethnic groups. One of the major risk factors identified for cardiovascular disease is elevated blood cholesterol and triglyceride levels. Desirably, an adult should have a total cholesterol level of <200 mg/dl. However, it has been found that over half of adult Americans have cholesterol levels above that target.

Policosanol is a group of active compounds composed primarily of five higher primary aliphatic alcohols—tetracosanol (C24), hexacosanol (C26), octacosanol (C28), triacontanol (C30), and dotriacontanol (C32). The various primary aliphatic alcohols found in policosanol have been previously isolated from a variety of materials, including sugar cane wax, beeswax, and rice bran wax.

Of these higher aliphatic alcohols, octacosanol and related primary alcohols have been sought as potential hypocholesterolemic agents. However, the amount of alcohols in oils and waxes is small. The content of octacosanol naturally occurring in plants usually ranges up to the tens or hundreds of parts per million—level of octacosanol in: apple peel (222 ppm); maize bran oil (9 ppm); rice bran oil (5 ppm); wheat bran oil (4 ppm); various nut oils (4-6 ppm). Accordingly, any attempt to provide compositions of these primary alcohols requires intense isolation and purification steps.

Sorkin, U.S. Pat. No. 6,197,832, discloses the use of a composition containing a combination of policosanol and phytosterols (1:3.2 ratio, respectively) to synergistically lower serum cholesterol levels. The policosanol used is isolated from any of a variety of sources, including rice bran wax, and contains 23-33% of total policosanols, with octacosanol being present at a level of about 5.6% of the total composition.

Commercially available policosanols have also been available containing 90-95% policosanols, at a level of octacosanol of about 50-60%. These have been marketed as LESSTANOL (available from Garuda International, Inc), and BIOCOSANOL 50 (available from Cyvex Nutrition, Inc.), particularly for their effect on lowering serum cholesterol levels.

Because of its presumed potential therapeutic effects, efforts have been concentrated on providing policosanol compositions containing higher levels of octacosanol, such as that found in policosanol derived from sugar cane wax (about 60% octacosanol). However, such attempts become increasingly costly due to the processing required and/or the starting materials required. There is a need for a policosanol composition of high purity (>90% policosanols) that is as effective as, or more effective than, the conventional compositions containing 50-60% octacosanols, relative to lowering serum cholesterol, but can be obtained from another source other than sugar cane wax and is less costly to produce.

### SUMMARY OF THE INVENTION

Accordingly, one object of the present invention is to provide a policosanol composition containing at least 90% policosanols, with moderate levels (25-35%) of octacosanol, having improved serum cholesterol lowering activity compared to compositions containing 50-60% octacosanol.

A further object of the present invention is to provide a method for treatment of hypercholesterolemia using this composition that is even more effective at lowering serum cholesterol level than compositions containing higher levels of octacosanol.

These objects, and others, have been satisfied by the discovery of a composition consisting essentially of at least 90% policosanols obtained from rice bran wax, wherein the policosanols comprise from 25-35% octacosanol, and its use in treating hypercholesterolemia in a subject in need thereof.

### DETAILED DESCRIPTION OF THE INVENTION

The present invention relates to a composition consisting essentially of at least 90% policosanols obtained from rice bran wax, and wherein the policosanols comprise from about 25-35% of octacosanol.

The present composition is obtained from rice bran, preferably from refined rice bran or purified, food grade, rice bran wax. While the product can be produced using a variety of processes involving extraction from the rice bran, followed by various crystallization/recrystallization and/or distillation steps, the preferred process used to obtain the composition of the present invention involves saponification of the rice bran using aqueous base (such as alkali metal hydroxide or alkaline earth metal hydroxide, preferably about 10-20% by weight of alkaline earth metal hydroxide, such as Ca(OH)₂, based on total weight of rice bran); extraction of the saponified mixture (after water has been removed by heating), using an alcohol solvent, such as ethanol, propanol, butanol, etc; recrystallization of the extracted product to increase the concentration of octacosanol to the desired levels; a first solvent removal step using heat to drive off the alcohol solvent; a second solvent removal step involving redissolving the product from the first solvent removal step, then using a thin-film vaporizer to remove the solvent; and molecular distillation of the material from the second solvent removing step, from 1 to 5 distillations as needed to reach the desired level of policosanols and octacosanol. The levels of the ingredients can be monitored during the process using conventional techniques such as chromatography (liquid, high-pressure liquid, gas-liquid, etc). Alternatively, the product may be prepared by a process of supercritical CO2 liquid extraction, followed by molecular distillation and crystallization. The process is performed with the number of recrystallizations and distillations being sufficient to concentrate the policosanols to a level of at least 90%, more preferably at least 93%, most preferably at least 95% of the total composition, as well as to raise the level of octacosanol to the desired range. The concentration of policosanols can be readily monitored using a variety of conventional laboratory techniques, such as high-pressure liquid chromatography (HPLC) or gas-liquid chromatography (GLC).

Once the product is obtained, it can be transformed into a powder form, using any conventional powder formation process, such as spray powder formation.

The composition of the present invention has a preferred component content, with respect to the five major aliphatic alcohols as follows:
Tetracosanol 0.5 to 15%
Hexacosanol 5 to 35%
Octacosanol 25 to 35%, preferably from about 28 to about 32%, more preferably about 30%
Triacontanol 12 to 49%
Dotriacontanol 2 to 17%.

Within the context of the present invention, the term “about” indicates the experimental accuracy to which the content of a component can be determined using conventional liquid chromatography/HPLC/GLC techniques for the given component, preferably within 1-2% of the stated value or range.

The composition of the present invention can be administered in any suitable orally administrable form. Suitable forms include powder, tablet, capsule, solutions, etc. The preparation of such administration forms is well known in the art and can also include conventional tableting auxiliaries or excipients. Preferably the composition is administered in the form of a soft-gel capsule or hard-shell capsule.

The dosage of the present composition can be administered once a day or in a multi-dose per day regimen. The total daily dosage is a dosage sufficient to lower total serum cholesterol. With the present composition, this is preferably from 10-20 mg/day for an average human adult. The dosage is preferably a single dose, which can be taken at any time of the day, but, if desired, can be divided into equal or unequal administrations during the day. If unequal dosages are used, the dosages should be adjusted to provide an approximately equal level of the composition's active ingredients in the bloodstream throughout the day, with larger dosages being provided when the time between doses is higher and smaller dosages being provided when the time between doses is shorter. The level of reported side effects for policosanol products in general is very low, with side effects that are encountered being found to be dosage independent.

The present inventors have found that administration of the present composition to adult humans gives a significantly greater reduction of total serum cholesterol and low-density lipids (LDL), compared to administration of the conventional policosanol products containing 50-60% octacosanol, when both are administered at the same total dosage level of policosanols. This provides the present composition with the potential to provide not only cost savings on the ability to use rice bran wax compared to other types of starting materials, but also provides the ability to lower total dosage levels while maintaining the overall decrease in total serum cholesterol and LDL, relative to the dosages used of the conventional 50-60% octacosanol products.

The reduction of total cholesterol and LDL found using the present composition was very surprising, considering the conventional position that the primary hypocholesteremic agent in policosanols is octacosanol. Thus, the previous efforts in the area have usually been to maximize the concentration of octacosanol relative to the other constituents in the policosanols product. The present composition, however, has cut the level of octacosanol roughly in half, but has resulted in a reduction in total serum cholesterol after an 8 week program of administration of approximately 40% greater reduction than the reduction using a 60% octacosanol containing product, with a reduction in LDL in the same study of approximately 66% greater reduction than the reduction using the 60% octacosanol containing product.

### EXAMPLES

Having generally described this invention, a further understanding can be obtained by reference to certain specific examples which are provided herein for purposes of illustration only and are not intended to be limiting unless otherwise specified.

A study was conducted to demonstrate the effectiveness of the present policosanols composition containing approximately 30% octacosanol, compared to a conventional composition containing 60% octacosanol, in controlling Type II hypercholesterolemia. The study included an 8 week open label study with 20 patients in each group. The two groups would receive either the present invention 95% policosanols composition containing approximately 30% octacosanol or a conventional 95% policosanols composition containing approximately 60% octacosanol. The patients were dosed at 10 mg/day with their composition, as a single dose. The subjects in each group had total cholesterol levels of 190-250 mg/dl. After the study, the subjects in both groups combined showed approximately 12% reduction in total cholesterol, and approximately 27% reduction in LDL.

Most surprisingly, the subjects that received the present policosanols composition having 30% octacosanol showed a reduction in total cholesterol that was approximately 40% greater reduction than the reduction seen using the 60% octacosanol product, and a reduction in LDL that was approximately 66% greater reduction than the reduction seen using the 60% octacosanol product.

Obviously, additional modifications and variations of the present invention are possible in light of the above teachings. It is therefore to be understood that within the scope of the appended claims, the invention may be practiced otherwise than as specifically described herein.

1. A composition consisting essentially of at least 90% policosanols obtained from rice bran wax, wherein the policosanols comprise from about 25 to about 35% of octacosanol.

2. The composition of claim 1, wherein the level of policosanols is at least 93%.

3. The composition of claim 2, wherein the level of policosanols is at least 95%.

4. The composition of claim 1, wherein the policosanols comprise from about 28 to about 32% of octacosanol.

5. The composition of claim 1, wherein the policosanols comprise about 30% of octacosanol.

6. The composition of claim 1, wherein the policosanols comprise from 0.5 to 15% of tetracosanol, from 5 to 35% of hexacosanol, from 25 to 35% of octacosanol, from 12 to 49 of triacontanol, and from 2 to 17% of dotriacontanol.

7. The composition of claim 1, wherein the level of policosanols is at least 95% and the policosanols comprise about 30% of octacosanol.

8. A method for treatment of hypercholesterolemia, comprising administering to a subject in need thereof an effective amount of a composition consisting essentially of at least 90% policosanols obtained from rice bran wax, wherein the policosanols comprise from about 25 to about 35% of octacosanol.

9. The method of claim 8, wherein the level of policosanols is at least 93%.

10. The method of claim 9, wherein the level of policosanols is at least 95%.

11. The method of claim 8, wherein the policosanols comprise from about 28 to about 32% of octacosanol.

12. The method of claim 8, wherein the policosanols comprise about 30% of octacosanol.

13. The method of claim 8, wherein the policosanols comprise from 0.5 to 15% of tetracosanol, from 5 to 35% of hexacosanol, from 25 to 35% of octacosanol, from 12 to 49 of triacontanol, and from 2 to 17% of dotriacontanol.

14. The method of claim 8, wherein the level of policosanols is at least 95% and the policosanols comprise about 30% of octacosanol.

15. The method of claim 8, wherein the composition is in a form selected from the group consisting of powders, tablets, capsules, and solutions.

16. The method of claim 8, wherein the composition is in a form selected from the group consisting of hard shell capsules and soft-gel capsules.

17. The method of claim 8, wherein the effective amount is from about 10 mg to about 20 mg per day.

18. The method of claim 8, wherein the administration is performed as a single dose per day.

19. The method of claim 8, wherein the administration is performed as multiple doses per day.

20. The method of claim 19, wherein the multiple doses per day are each equal in size to one another.
